(19) United States
(12) Patent Application Publication
(10) Pub. No.: US 2026/0165759 A1
(43) Pub. Date: Jun. 18, 2026

(54) VERTEBRAL NAIL: SPINE SEGMENTAL FIXATION IN COMPROMISED BONE

(71) Applicants: Dimitriy KONDRASHOV, Sherman Oaks, CA (US); Victor UNGUREAN, Chisinau (MD)

(72) Inventors: Dimitriy KONDRASHOV, Sherman Oaks, CA (US); Victor UNGUREAN, Chisinau (MD)

(21) Appl. No.: 19/466,002

(22) Filed: Jan. 30, 2026

Related U.S. Application Data
(63) Continuation-in-part of application No. PCT/US2024/038625, filed on Jul. 18, 2024.
(60) Provisional application No. 63/516,770, filed on Jul. 31, 2023.

(51) Int. Cl. A61B 17/84

(52) U.S. Cl. CPC A61B 17/846

(57) ABSTRACT

A vertebral or pelvic nail device is provided that includes a body including one or more protrusions extending from the body or the body defining one or more negative features into the body. The body may have a tapered end for insertion into a patient. Examples include a head having a fastener adapted to receive a connector. The body may have a first portion and a second portion, the first portion extending at an oblique angle with respect to the second portion. The tapered end of the body may be at an opposite end of the body from the head. Also provided are systems and kits that include a nail and a connector, such as an offset bar, rod, plate or rail, that may be affixed to the fastener of the head of the body.

### CROSS-REFERENCE TO RELATED APPLICATIONS

This application is a Continuation-in-part of PCT/US2024/038625 filed on Jul. 18, 2024, which claims the benefit of U.S. Provisional Patent Application 63/516,770, filed on Jul. 31, 2023, the disclosures of which are herein incorporated by reference in their entireties.

### FIELD

The present invention relates generally to the field of systems and methods for making connections between bones or bone parts, and more specifically, a nail device for connecting bones or placing an anchor for example into the spinal column or pelvis.

### BACKGROUND

Spinal fixation and instrumentation are crucial elements of spine surgery. These techniques are used for the treatment of degenerative, traumatic, infectious, and neoplastic conditions. Achieving solid fixation of spinal segments is crucial for obtaining a solid spinal fusion, thereby addressing neural compression, spinal instability or deformity.

Pedicle screws are the gold standard for segmental spinal fixation of the thoracic, lumbar, or sacral spine. While pedicle screws provide 3-column fixation and have established an excellent track record across several decades, they are likely to achieve suboptimal purchase in osteoporotic bone, as well as in cases of revision spine surgery where the bone has been compromised by prior fixation. Osteoporosis affects both cortical and cancellous bone quality. Prior instrumentation in the setting of revision surgery compromises the bone stock available for additional instrumentation.

Current strategies to deal with compromised bone include using bone cement for screw augmentation and using screws with a greater length and a larger diameter.

However, both of these strategies are accompanied by significant downsides. For bone cement augmentation, concerns include the possibility of cement extravasation into both the adjacent vasculature and the spinal canal, causing neurological or vascular compromise. Another potential issue is the revisability of a cemented screw, especially in the case of possible infection. Upsizing the screw faces the obstacle of the limited availability of screw sizes, which typically go up to 8 to 8.5 mm in diameter. In addition, increasing the outer diameter of a screw is limited by the inner cortical diameter of the pedicle.

Using 8 to 8.5 mm screws is typically reserved for fixation in S1 and sometimes L5 segments. However, in the case of a prior fixation with an 8 to 8.5 mm screw, revision options are very limited. Thus, there is a need to develop a device and methods to solve the problem.

The present application addresses problems associated with conventional devices.

### SUMMARY

Embodiments of the present spinal or pelvic nail devices and systems disclosed in this application are tailored for medical purposes, showcasing innovative structural features that improve functionality and ease of use.

Embodiments of the present vertebral or spinal nail devices can be used for spine or pelvic instrumentation, and can be crafted e.g., with a curved body, resembling a pedicle screw trajectory and/or mimicking the natural curve of the thoracic or lumbar vertebrae or a patient, in contrast to a conventional pedicle screw, which is straight.

Durable materials including, but not limited to commercial titanium (such as pure commercial titanium), titanium alloy, cobalt chrome one alloy or stainless steel are utilized, while the tulip component aids in securing a spinal rod or rail and connecting the spinal rod to other pedicle screws, hooks or similar curved bone anchors.

In embodiments of the present nails having a head, the head may be made of the same or a different material than the body of the nail. Examples showing a head of a different material may be seen for example in FIGS. 7, 11 and 12. Examples showing a head of the same material as the body may be seen for example in FIGS. 8-10

Attributes of example embodiments can include for example, serrated ridges or other protruding features for better grip, pushing torque and pullout strength, cutouts for bony ingrowth, curved shaft, features to minimize neural structure impingement, and customizable ridge configurations. Other attributes may include negative features such as slots, holes, crevices, or grooves defined by the body of the nail. Other variations of surface topography are also contemplated. By incorporating precise manufacturing techniques and adaptable design elements, the present vertebral nail devices improve spinal fixation in compromised bone, and enhance surgical performance and patient outcomes.

### BRIEF DESCRIPTION OF THE DRAWINGS

FIG. 1 is a perspective view of an embodiment of an example nail in accordance with non-limiting embodiments of the present application.

FIG. 2 is an illustration of an embodiment of a side view of an example nail in showing the body and head in accordance with non-limiting embodiments of the present application.

FIG. 3 is an illustration of an embodiment of the rear view of an example nail in accordance with non-limiting embodiments of the present application.

FIG. 4 is an illustration of an alternative embodiment of an example nail in accordance with non-limiting embodiments of the present application, with ridges.

FIG. 5 is an illustration of an alternative embodiment of the present application with slots.

FIG. 6 is an illustration of an alternative embodiment of a system of the present application with the offset rod connected to a nail.

FIGS. 7-23 depict non-limiting example embodiments of example nails provided herein

FIG. 24 depicts an illustration of a tulip with a threaded set screw that can attach to a threaded hole inside of an example nail, according to non-limiting example embodiments.

### DETAILED DESCRIPTION

Hereinafter, embodiments in the example embodiment will be described as follows with reference to the accompanying drawings. Items described in the singular herein may be provided in plural, as can be seen, for example, in the drawings. Thus, the description of a single item that is provided in plural should be understood to be applicable to the remaining plurality of items unless context indicates otherwise.

As used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. When used herein to join a list of items, “and” denotes “all of the items of the list.”

The use of “at least one” or “one or more” followed by a list of elements refers to only one of the elements as well as any and all combinations of the elements. The term “and/or” (which may be abbreviated as “/”) also will be understood to connote this scope. For example, each of the phrases “at least one of A or B,” “at least one of A and B,” and “at least one selected from the group of A and B,” refer to only A, only B and the combination of A and B (as well as allowing for additional unlisted elements if the limitation is open ended and context allows).

It will be understood that when an element is referred to as being “connected” or “coupled” to or “on” another element, it can be directly connected or coupled to or on the other element or intervening elements may be present. In contrast, when an element is referred to as being “directly connected” or “directly coupled” to another element, or as “contacting” or “in contact with” another element (or using any form of the word “contact”), there are no intervening elements present at the point of contact.

While embodiments are described herein in detail in relation to one or more embodiments, it is to be understood that this disclosure is illustrative and exemplary of the present disclosure.. The detailed disclosure herein of one or more embodiments is not intended, nor is to be construed, to limit the scope of the present application.

Each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Wherever possible, the same reference numbers are used in the drawings and the following description to refer to the same or similar elements. While many embodiments of the disclosure may be described, modifications, adaptations, and other implementations are possible.

Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

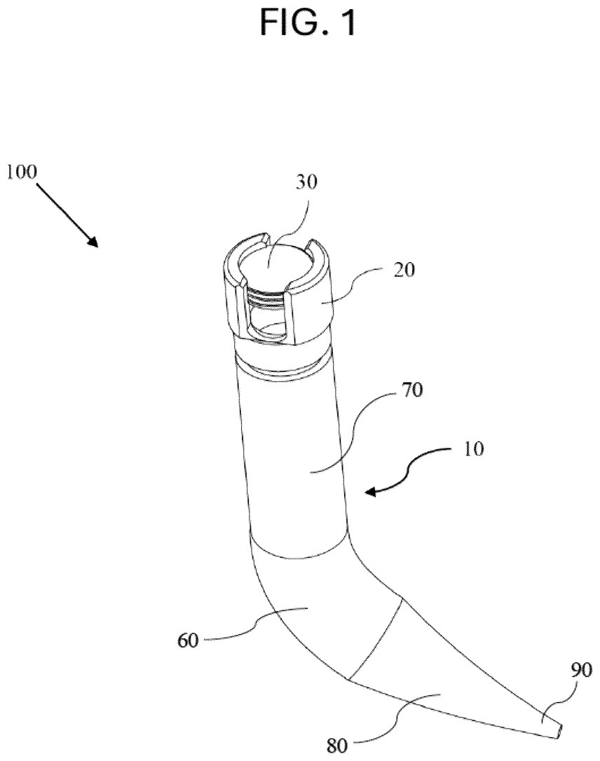

A nail device 100 (also referred to as a “nail”) is disclosed herein. Also included are systems and kits including the present nail devices. Examples are illustrated in FIGS. 1-6. Non-limiting examples include a body 10 and a head 20.

In example embodiments, the head 20 is attachable to the body 10 to create a nail device 100. Alternatively, the head 20 can be an integral part of the body 10 and be manufactured together as one piece. In example embodiments, the head 20 may be or include a fastener, such as a tulip (shown as the head 20 e.g. in FIG. 1), which fastener may be configured to link a connector 25 to the nail 100. The head 20 (e.g. fastener) may be rigid or may be adjustable or rotatable in position with respect to the body, and may also be lockable to a desired position.

According to example embodiments, a vertebral pedicle nail device is provided, which may be used for spine instrumentation in the sacral, lumbar, thoracic spine or pelvis. In particular, example nail devices may include a body having a tapered end 90, the body including one or more protrusions (e.g. ridges 46 or spikes 45) extending from the body, or the body defining one or more negative features (e.g. 47) into the body, and the body having a tapered end; and optionally a head, which head may include or be a fastener configured to receive a connector.

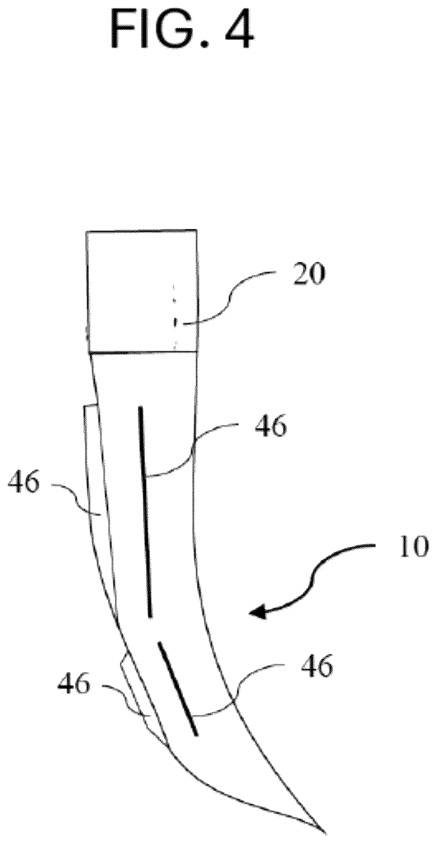

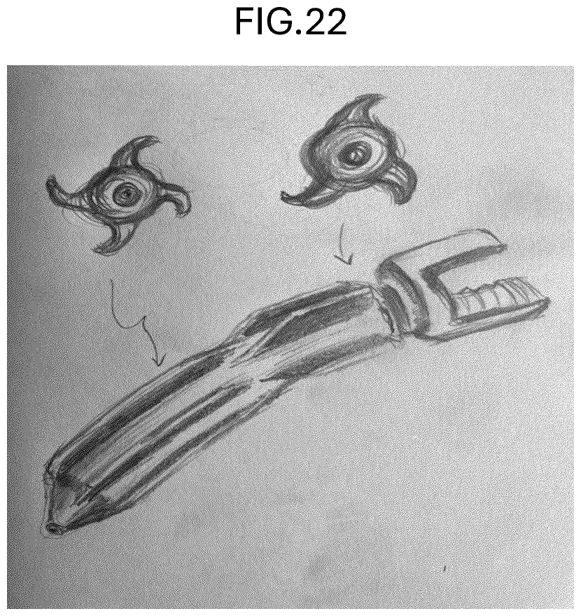

The one or more protrusions may for example, include one or more ridges extending outward from the body, the ridges being angled, such as at oblique or perpendicular angles relative to the body and/or may have a curved portion with or without angles as shown for example in FIG. 4. FIG. 22 also depicts a non-limiting example embodiment having ridges.

The tapered end 90 of the body 10 may be for example, at an opposite end of the body from the head 20. The tapered end may or may not come to a point at the end and may be configured to be insertable into bone.

In example embodiments, in the present nails or vertebral nail devices, the body 10 may include a curved shape or portion 60. In example embodiments the curved portion may be configured to connect, bridge or transition a first portion 70 of the body and a second portion 80 of the body. The first portion 70 of the body may extend in a first direction, and a second portion 80 of the body may extend in a second direction that is at an oblique or perpendicular angle with respect to the first direction. Extending in a first direction may be defined to mean that a central axis of the first portion of the body extends in the first direction. Extending in a second direction may be defined to mean that a central axis of the second portion of the body extends in the second direction. The curved portion 60 may be a portion of the body that is curved or angled to connect the first portion 70 and second portion 80 as depicted for example in FIG. 1. The first portion 70 may be for example cylindrical or substantially cylindrical and may have a constant or substantially constant cross-sectional diameter through the length of the first portion 70, or the diameter may vary, such as narrowing along the length of the first portion 70.

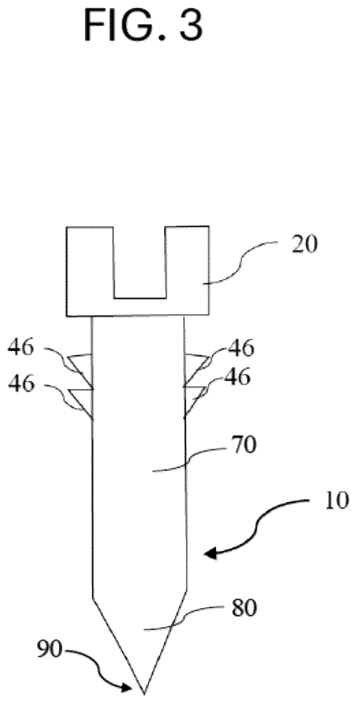

In alternative embodiments, a first portion 70 of the body may extend to the second portion 80 of the body such that both portions extend in the same direction without a curved portion therebetween, as shown for example in FIG. 3.

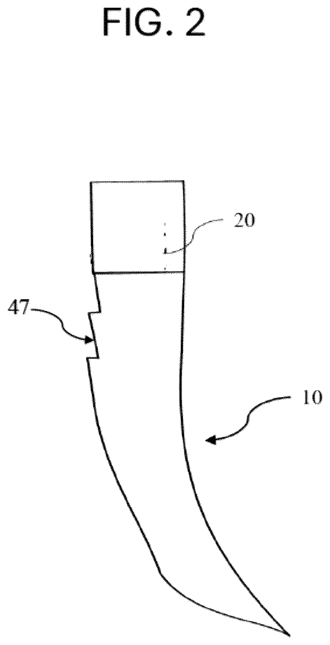

In example embodiments, the body 10 may extend in one continuous curved portion as shown for example in FIG. 2.

In example embodiments, the diameter of the first portion 70 of the of the body 10 may be wider at an end adjacent to the head of the nail and may narrow as it approaches an opposite end of the nail from the head. In other example embodiments, the diameter of the entire body 10 may be wider at an end adjacent to the head of the nail and may narrow as it approaches the tapered end 90 of the body 10 (as depicted e.g., in FIG. 2). The narrowing of the diameter of the body 10 may be gradual as shown for example in FIG. 2, or the diameter may be constant or relatively constant through the extending length of a first portion 70 of the body 10, and then narrow more sharply in a second portion 80 of the body to the tapered end 90 as shown in FIG. 3.

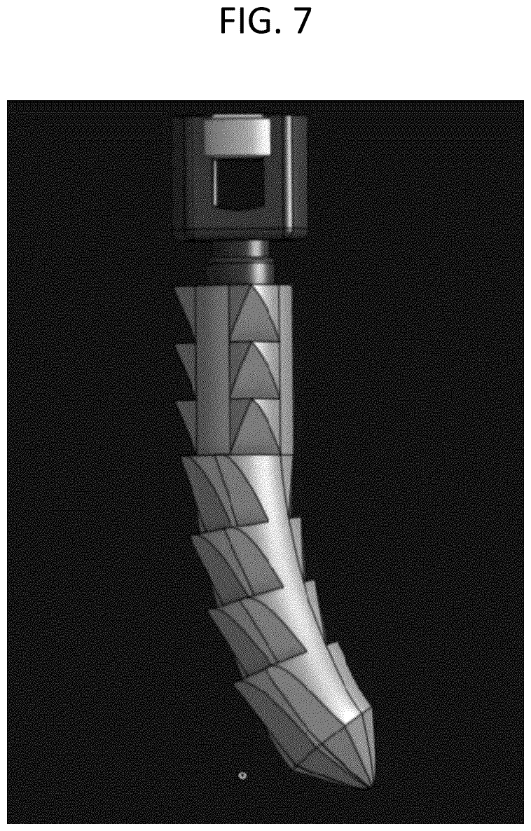

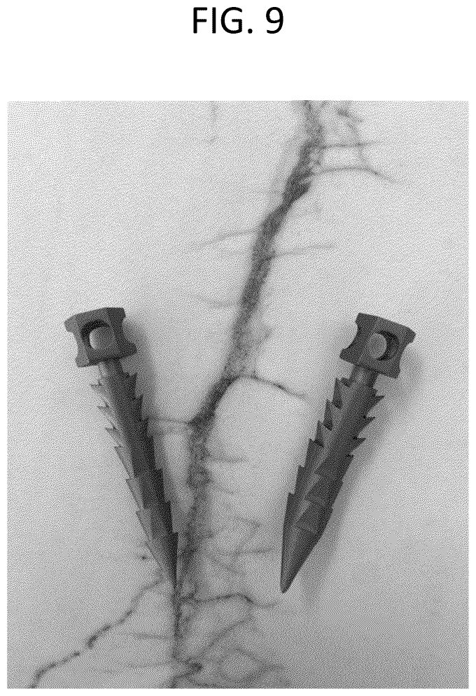

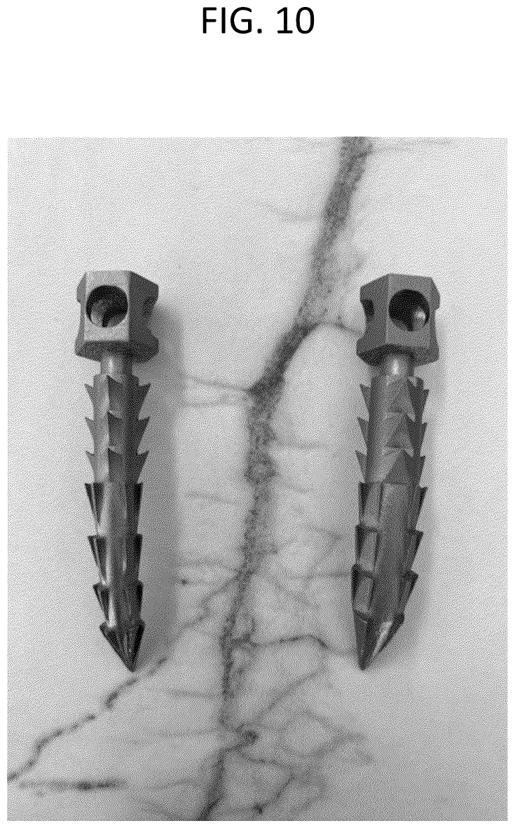

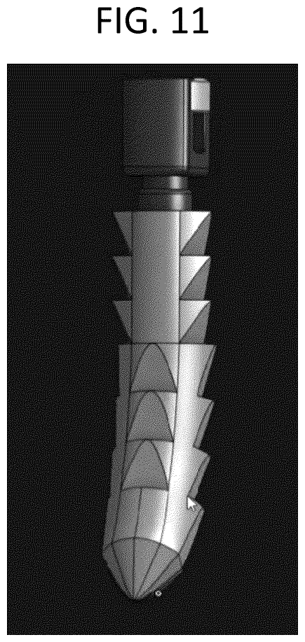

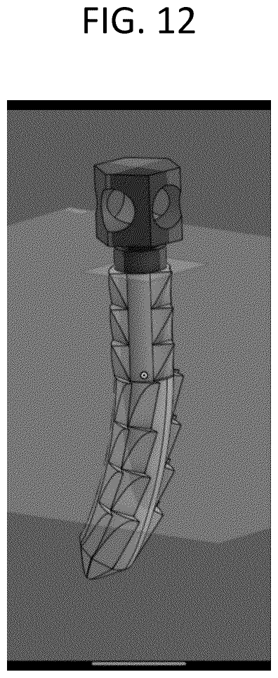

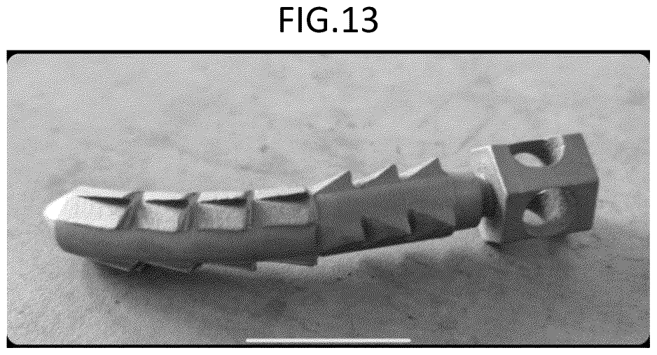

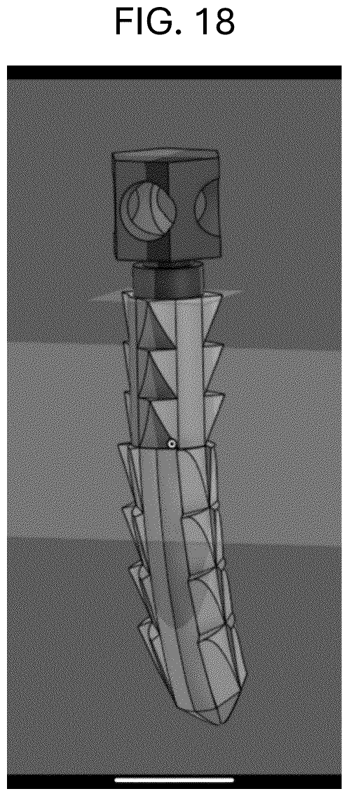

As shown in FIG. 2, one or more negative features, such as cut-outs 47 may be included on or in the body, which may be considered either a cut-out of the body, or may be considered a spike that is part of the body and extends from the body. Additional examples of such cut-outs are also depicted for example in embodiments shown in FIGS. 7-13 and 18-19.

In example embodiments, the head may include a fastener, such as a tulip. The head may be integral with the body or it may be connected to the body, such as threadedly or rotatably connected to the body. In example embodiments, at least a portion of the body has a cylinder configuration with varying curvature radii along a length of the body. In example embodiments, the head is threadedly connected to the body, or connected by another method. In example embodiments, the body may define one or more negative features. The body may include one or more spikes.

Example embodiments also include a vertebral nail device including a body including a cylindrical portion and a tapered end, the body including one or more protrusions extending outward from the body and/or one or more negative features into the body, which are defined by the body. For example, negative features may include cut-outs, slots, holes, crevices or grooves. The cylindrical portion of the body may have fixed or varying curvature along a length of the cylindrical portion of the body. The vertebral nail device also includes a head, which may be threadedly connected to the body, the head including a fastener configured to receive a connection. The head may have threads configured and dimensioned to mate with threads of the body, such that the head and body my be screwed together.

An example head may include a tulip, and the tapered end of the body may be at an opposite end of the body from the head. In example embodiments, the body may have a curved shape or a curved portion. In example embodiments the tulip of the head may be fixed or rotatably connected to the body. In example embodiments, the body includes one or more ridges that are angled relative to the cylindrical portion of the body. The head, such as a tulip, may be configured to receive a connector, such as an offset bar, rod or rail, to affix the connector to a top of the body.

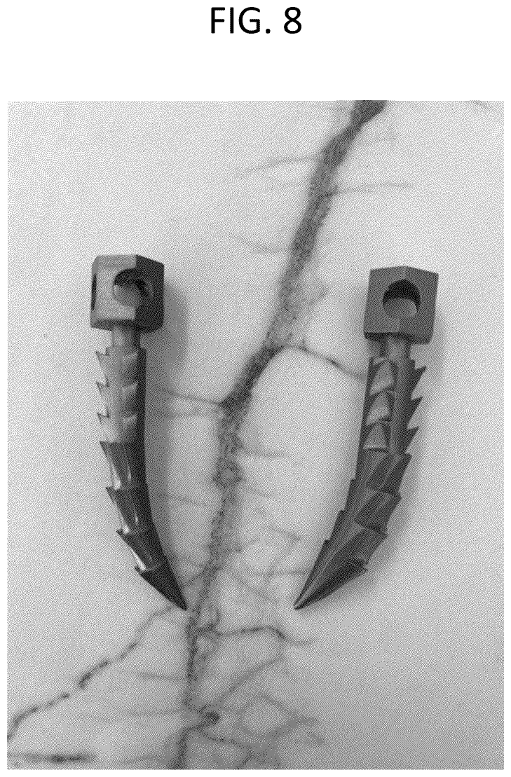

Examples of the head may include other configurations of fasteners other than a tulip. For example, the head may include other configurations with one or more openings adapted to be able to receive a connector therein. Non-limiting examples of other configurations of a fastener are depicted in FIGS. 8-10, 12-13, and 18-19. These figures also show examples in which a head or fastener may be configured in a shape such that viewed from above, the head may have angled sides (such as a hexagon), which may allow one to use certain tools (e.g. having a hex screwdriver head) adapted to to assist in insertion and/or positioning of the nail or the head, for example to turn the head to a desired location for insertion of a connector therein.

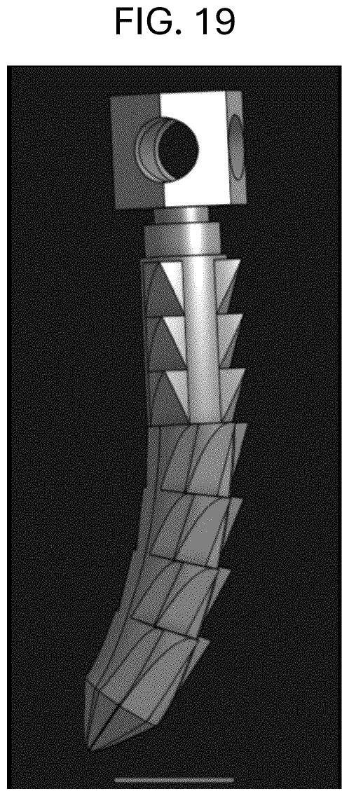

The opening(s) in example fasteners may have a circular, cylindrical or other shape suitable for receiving a connector that may be inserted for example from the side or top of the opening. For example, in the case of a tulip fastener, a connector may be inserted from above the tulip. Other configurations may include openings in which a connector may be inserted from the side, such as shown for example in FIGS. 8-10. The connector may be directly inserted from the side or top, or it may be inserted at an angle with respect to the head. As can be seen for example in FIG. 19, multiple holes may be present to allow insertion of one or more connectors into or through the head from various positions or sides of the head.

The cylindrical portion of the body may be hollow and may be configured to allow guided insertion of the vertebral nail device into a patient, over a guidewire.

In a particular embodiment, the nail device 100 may comprise a curved body 10, such as a curved metallic vertebral body nail that may be shaped to mimic the trajectory of a pedicle screw and the natural curvature of the vertebral body of a patient into which the nail device is to be inserted.

In example embodiments, the body 10 can be constructed from materials including titanium alloy, pure commercial titanium, stainless steel, or cobalt chrome.

The head 20 of the nail device 100 may be or include a fastener, such as a mobile or fixed tulip (also labeled number 20 in the figures, the tulip being the head depicted in the figures) according to non-limiting example embodiments.

According to examples, the head 20 is a fastener such as a tulip head that threads into a threaded hole on top of the curved body of the nail. The particular fastener may be selected for example based on the intended use of the present nails.

In non-limiting example embodiments, the head may be for example, a tulip—mono or polyaxial, for example in spinal applications (e.g., sacral, lumbar or thoracic).

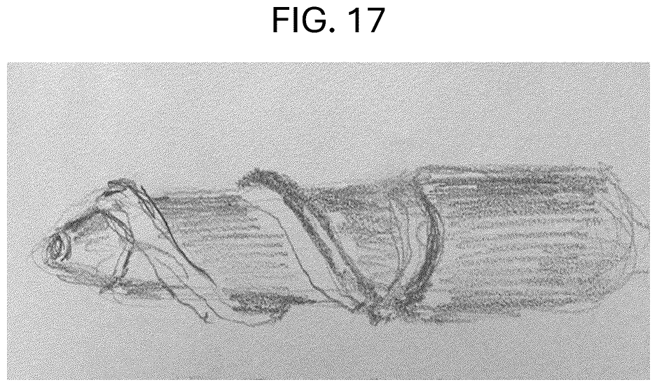

According to alternative embodiments, for example in the case of a nail that is configured for use for SI fusion (Sacroiliac joint) in the pelvis, nails provided herein may not include a head or a head may not include a fastener. For example, a tulip may not be used, for example as shown in FIG. 17.

A tulip 20 may be used for example, in medical spine segmental fixation as a component used in spinal instrumentation systems. It facilitates the connection between the spinal rod and pedicle screws or anchors. The tulip's design allows for secure attachment and alignment of the spine during surgery. It plays a vital role in maintaining stability and promoting fusion of the vertebrae.

Some variations of embodiments of the nail device 100 may incorporate the body 10, a separate tulip 20, and a separate set screw 30 for assembly. The tulip 20 on the head can be fixed or rotatable to facilitate connection of segments with a rod or rail.

Manufacture of the nail device 100 can involve machining or 3D printing. The device 100 is designed for insertion during surgery on a patient along a prepared path, following axial force application, rather than rotation like a pedicle screw.

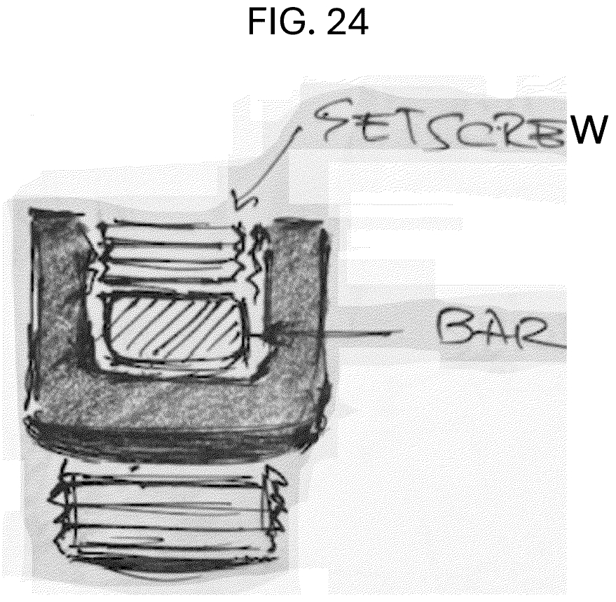

FIG. 24 depicts, inter alia, a tulip head as a fastener, which has a connector (bar) therein and a set screw over the bar to hold the bar in place. As can be seen in FIG. 24, the tulip may have internal threading adapted to receive a threaded set screw to hold the bar in place within the tulip. As shown in FIG. 24 the tulip head may also include a threaded bottom portion that can attach to a threaded hole inside of a body of an example nail, according to non-limiting example embodiments. It is contemplated that a head, such as a tulip head may connect to a body of a nail by other methods which may allow the tulip or other head to be rotatable with respect to the body in non-limiting example embodiments.

Examples of the body 10 of the nail device 100 may be e.g. cylindrical or conical, with varying curvature radii. The nail device can have an inside hollow channel—cannulation—to allow guided insertion of the nail device over a guidewire.

As shown in FIGS. 2-4, the body 10 may include multiple ridges or other protruding elements (shown for example as ridges 46 in the figures, although other configurations are contemplated) with or without serrations along the convex side to enhance grip, while a smaller feature on the concave side reduces the risk of neural structure impingement.

Some example embodiments feature three or more ridges 46 with serrations near the tulip 20 and tip, which can be straight or slightly angled (e.g. oblique angles—see e.g. FIG. 3) or curved (see e.g. FIG. 4) relative to the body 10.

In example embodiments, a maximum cross-sectional diameter of the nail device 100 for example at a location along the length of the body 10 that has a largest diameter may be 5 mm, or from 1 mm to 20 mm, or 2 mm to 15 mm or 0.5 mm to 6 mm. Other example embodiments may be from 1 mm to 5 mm or 2 mm to 4 mm. A ridge 46 height (or length) may be 1 mm or greater, or 1 mm to 5 mm, or 1.5 mm to 4 mm, or 0.1 mm to 1 mm, or 0.2 mm to 0.8 mm According to example embodiments, a ridge may have a height or length of less than 1 mm. Ridges may extend lengthwise down the body of the nail or a portion thereof. Alternatively, ridges may extend at least partially around a circumference of a body of the nail.

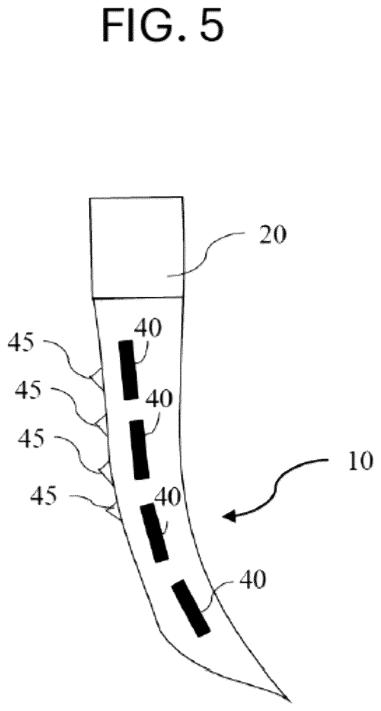

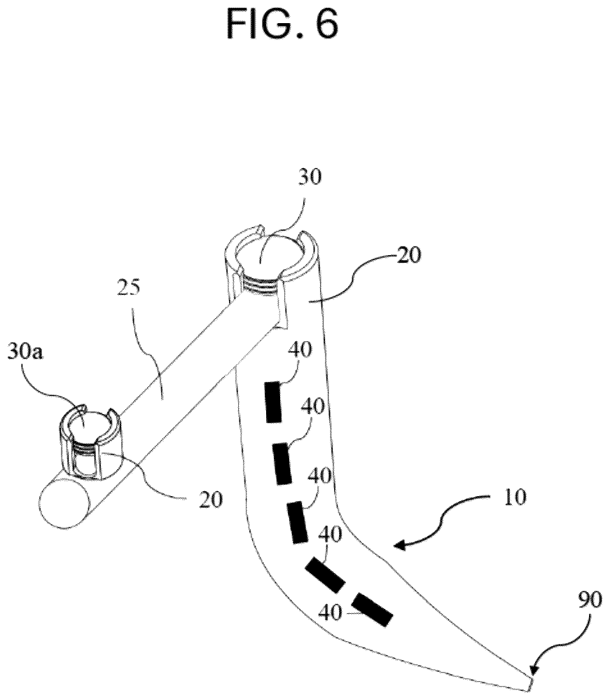

In an alternative embodiment, as shown in FIGS. 5-6, the body 10 may incorporate one or more slots or openings 40, which may be configured in size and/or shape to enhance bone ingrowth or osseointegration or to allow bone grafting prior to implantation of the nail.

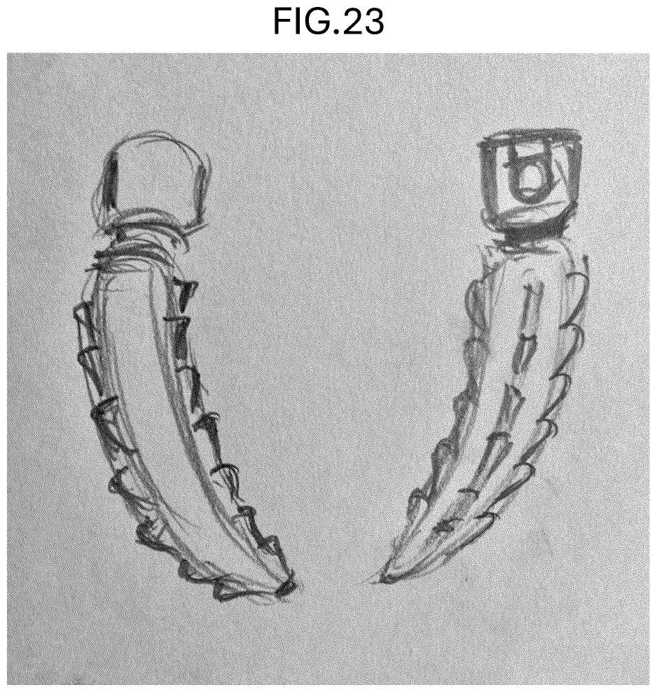

Example embodiments of the body 10, as shown in FIGS. 5 and 23, may further comprise multiple spikes 45. The slots or openings 40 are non-limiting examples of negative features. Other negative features may include holes, crevices, cut-outs, grooves or other indentations or openings in the body.

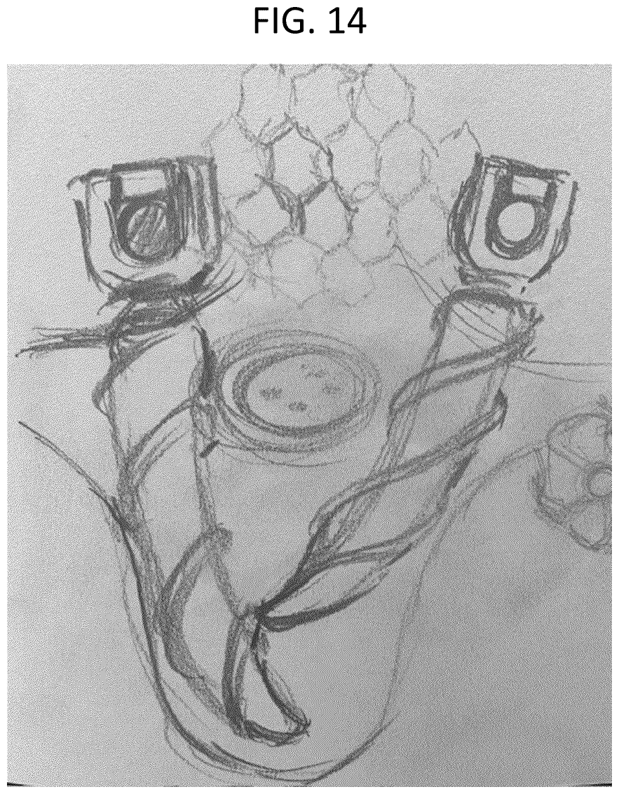

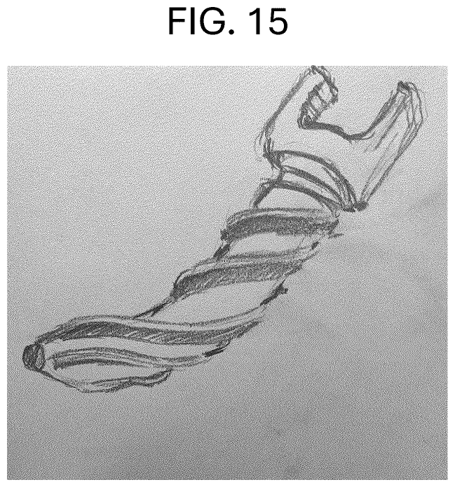

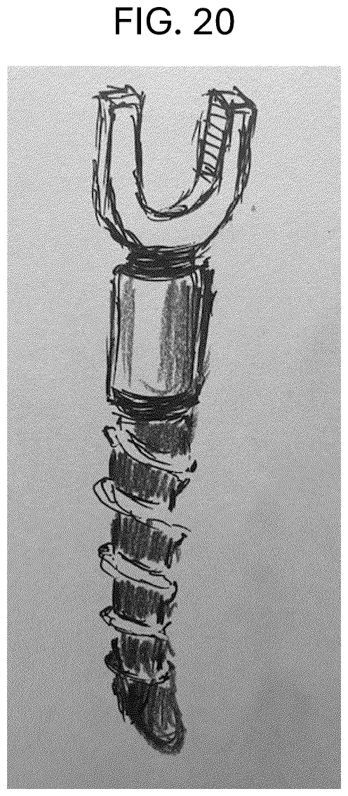

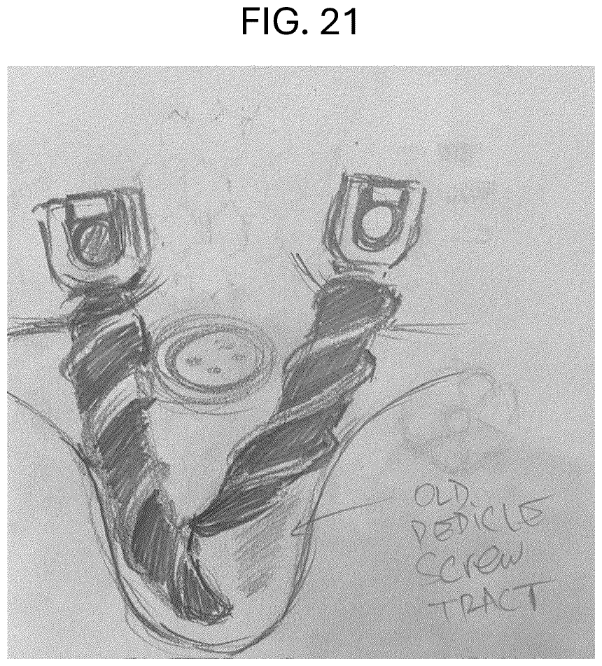

Further example embodiments may include one or more protrusions for example in a helical configuration that may extend along at least a portion of the length of the nail, or along the entire length of the nail or the body of the nail. Example embodiments of such helical protrusions (which may be added onto the body, or which may be part of the body itself, such as made up of the same material as the body) are depicted for example in FIGS. 14-17 and 20-21.

Additionally, as shown in FIG. 6, further embodiments herein may be systems that include a nail device 100 as described herein, such as a vertebral nail, and a bar, rod, rail, plate or other connector, which may be used to attach multiple nails to one another or to attach a nail to a plate or other device, such as pedicle screw, plate or hook. By way of non-limiting example embodiment, the present systems may include a nail 100 as shown in FIG. 6 and an offset bar 25 affixed to the top of the nail, such as the head of the nail. For example, the nail may include a body and a head, and the head may be or include a fastener, such as a tulip 20, which fastener is configured to link the connector 25 to the nail 100. The connector 25 may be an offset or other bar, rod, rail, plate or any other connector. According to non-limiting example embodiments, the connector 25 may include one or more additional fastener 20 thereon or as part of the connector 25, which may be adapted to be able to receive a further connector (as shown attached to connector 25 in FIG. 6. The additional fastener 20 may be for example a tulip, which may be configured to receive a second connector such as a rod, rail, bar, plate or other connector, which may be the same or different size as the first connector. In example embodiments, a second or more set screw 30a may be used to secure a second connector in the second fastener 20.

In example embodiments, the position of the fastener, such as a tulip 20 may be adjusted with respect to the body 10, to a desired position for positioning a bar, rod, rail, or other connector affixed to the nail.

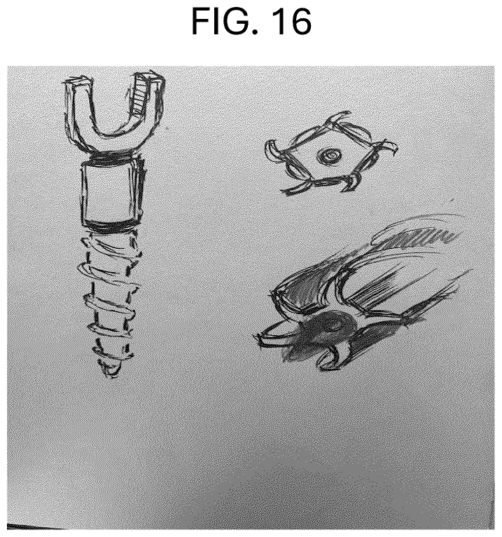

Further non-limiting example embodiments may include a body having a non-cylindrical cross-sectional shape. By way of non-limiting example, a portion or all of the body may have a polygonal shape, hexagon, star shape, or any other shape that may have curvature and/or may be more angular, etc. Non-limiting examples of a body having a non-cylindrical cross-sectional shape (or a portion having a non-cylindrical cross-sectional shape) may be be seen in FIG. 16, with the cross sections shown to the right of the example nail. The protrusions in example embodiments further emphasize the non-cylindrical cross-section.

Example embodiments include a system including a vertebral nail as set forth herein and a connector. In example embodiments the vertebral nail may include a body and a head. In non-limiting example embodiments, the body may have a tapered end and include one or more protrusions extending from the body or the body defines one or more negative features into the body. For example, the body including one or more protrusions (such ridges or spikes or helical protrusions or any other positive feature), a surface topography (created for example, by a coating or grooves etched into a surface or other surface irregularities, or a texture on the surface, such as microfeatures), and/or negative features, such as slots, holes, crevices, or grooves, etc. The head may include a fastener, such as a tulip head configured to receive a connector. The tapered end of the body may be at an opposite end of the body from the head. Example systems may include a connector affixable to a top of the body of the vertebral nail, in which the fastener is configured to receive and connect the vertebral nail to the connector.

In example embodiments of the present systems, the fastener may be part of or affixable to a top of the body of the vertebral nail. In non-limiting examples, the head is, or includes a fastener such as a tulip configured to be able to receive and connect to the connector, such as an offset bar.

Example embodiments of the present nails, which may be used in the present systems may include a curved body 10 with surface topography, such as 3D printed features, rather than, or in addition to, ridges and/or spikes or other protrusion(s).

In example systems, the tulip may be rotatably connected to the body. In other example embodiments, the head of the vertebral nail is attached to the body of the vertebral nail, by internal threads of the head being configured and dimensioned to mate with external threads of the body.

Example systems may also include a set screw 30 that may be used for example to hold a connector (such as an offset rod) in the fastener of the head (such as a tulip).

Also provided are kits that include a nail provided herein and at least one additional component including instructions for use or insertion of the nail or system, one or more attachments, rods, plates, rails, bars, offset connectors, a set screw, guide wires, wires drills and other tools for insertion, placement, attachment or visualization of the present device(s) or system(s) into a patient.

Further kits provided herein may include multiple nails of varying sizes and/or shapes.

Also provided are methods that include placing a nail provided herein at a desired location in a patient in need of the nail, and securing the nail to bone of the patient. Example methods include preparing a straight or curved channel in the pedicle and body of the vertebra, that can be undersized or sized line-to-line with the dimensions of the vertebral nail to be implanted, The straight channel may be prepared by using a pedicle finder or awl and further enlarged by using taps of various diameter which may be solid or cannulated working over a guidewire. A curved path might be used by placing a curved undersized awl shaped similarly to the vertebral nail along the intended path of insertion. The path is palpated using a ball tip probe to make sure the walls of the channel and bottom are intact. The channel creation is followed by placement of the curved vertebral nail using malleting or any other method of applying axial force. The nail may be inserted over a guidewire. Following the proper seating of the nail a mono—or polyaxial tulip might be threaded into the threaded opening on top of the nail to allow for connection with other spinal anchors—either other vertebral nails, or pedicle screws, tulip plates or hooks. Then, a rod or rail would be placed into the tulip (tulips) and set screws placed and tightened to allow for secure connection and lock the polyaxiality.

Although the invention has been explained in relation to example embodiments, it is to be understood that many other possible modifications and variations can be made without departing from the spirit and scope of the invention.

What is claimed is:

1. A vertebral or pelvic nail device comprising:
a body having a tapered end, the body including one or more protrusions extending from the body or the body defining one or more negative features into the body, and
a head comprising a fastener configured to receive a connector,
wherein the tapered end of the body is at an opposite end of the body from the head.

2. The vertebral or pelvic nail device as claimed in claim 1, wherein the body includes a curved portion.

3. The vertebral or pelvic nail device as claimed in claim 1, wherein the head includes a tulip.

4. The vertebral or pelvic nail device as claimed in claim 3, wherein the tulip is rotatably connected to the body.

5. The vertebral or pelvic nail device as claimed in claim 1, wherein the head is integral with the body.

6. The vertebral or pelvic nail device as claimed in claim 1, wherein at least a portion of the body is a cylindrical with varying curvature radii along a length of the body.

7. The vertebral or pelvic nail device as claimed in claim 1, wherein the head is threadedly connected to the body.

8. The vertebral or pelvic nail device as claimed in claim 1, wherein the one or more protrusions include one or more ridges or spikes extending outward from the body, the ridges or spikes extending at an oblique or perpendicular angle relative to the body.

9. The vertebral or pelvic nail device as claimed in claim 1, wherein the body defines one or more negative features.

10. The vertebral or pelvic nail device as claimed in claim 1, wherein a first portion of the body extends in a first direction, and a second portion of the body extends in a second direction at an oblique angle with respect to the first direction.

11. A vertebral or pelvic nail device comprising:
a body including a cylindrical portion and a tapered end,
wherein the body includes one or more protrusions extending outward from the body,
wherein the body defines one or more negative features into the body, and
wherein the cylindrical portion of the body has a fixed or varying curvature along a length of the cylindrical portion of the body; and
a head threadedly connected to the body, the head comprising a fastener configured to receive a connector, wherein the head comprises threads configured and dimensioned to mate with threads of the body,
wherein the head comprises a tulip, and
wherein the tapered end of the body is at an opposite end of the body from the head.

12. The vertebral or pelvic nail device as claimed in claim 11, wherein the body has a curved shape.

13. The vertebral or pelvic nail device as claimed in claim 12, wherein the tulip of the head is at a fixed position with respect to the body.

14. The vertebral or pelvic nail device as claimed in claim 12, wherein the tulip of the head is rotatably connected to the body.

15. The vertebral or pelvic nail device as claimed in claim 12, wherein the body includes one or more surface irregularities that are angled relative to the cylindrical portion of the body.

16. The vertebral or pelvic nail device as claimed in claim 11, wherein the tulip is configured to receive and affix a connector to a top of the body.

17. The vertebral or pelvic nail device as claimed in claim 11, wherein the cylindrical portion of the body has a hollow configuration to allow guided insertion of the vertebral nail device into a patient, over a guidewire.

18. A system comprising:
a vertebral or pelvic nail device comprising:
a body having a tapered end, the body including one or more protrusions extending from the body or the body defining one or more negative features into the body, and
a head comprising a fastener configured to receive a connector,
wherein the tapered end of the body is at an opposite end of the body from the head; and
a connector affixable to a top of the body of the vertebral nail, wherein the fastener is configured to receive and connect the vertebral nail to the connector.

19. The system as claimed in claim 18, wherein the fastener is a tulip rotatably connected to the body.

20. The system as claimed in claim 18, further comprising a set screw.
